(19) United States
(12) Patent Application Publication
(10) Pub. No.: US 2021/0284622 A1
(43) Pub. Date: Sep. 16, 2021

(54) BIOACTIVE POLYMERS WITH ANTI=FOULING PROPERTIES FOR MEDICAL AND DENTAL USES

(71) Applicant: OREGON HEALTH & SCIENCE UNIVERSITY, Portland, OR (US)

(72) Inventors: Carmem Pfeifer, Portland, OR (US); Matthew Logan, Portland, OR (US); Alexander Kendall, Portland, OR (US); Ana Paula Piovezan Fugolin, Portland, OR (US); Steven Lewis, Portland, OR (US)

(21) Appl. No.: 17/191,552

(22) Filed: Mar. 3, 2021

Related U.S. Application Data
(60) Provisional application No. 62/985,781, filed on Mar. 5, 2020.

(51) Int. Cl. C07D 333/70; C07D 307/85; C07D 239/20; A61K 6/887; A61K 6/69

(52) U.S. Cl. CPC C07D 333/70; C07D 307/85; A61K 6/69; A61K 6/887; C07D 239/20

(57) ABSTRACT

The present invention provides polymers capable of inhibiting the growth of biofilms, including biofilms of Streptococcus mutans. The invention also provides monomers for use in the preparation of such polymers.

### GOVERNMENT SUPPORT

This invention was made with government support under R01 DE028757, R35 DE029083, and K02 DE025280 awarded by the National Institutes of Health. The government has certain rights in the invention.

### FIELD OF THE INVENTION

The present invention concerns novel compounds useful for the prevention of biofilms, particularly as incorporated into dental polymers.

### BACKGROUND OF THE INVENTION

Bioactive compounds useful in inhibiting the biofilm of S. mutans have been described in the article Structure-Based Discovery of Small Molecule Inhibitors of Cariogenic Virulence, Zhang et al., Scientific Reports, 7:5974, DOI:10.1038/s41598-017-06168-1, published online 20 Jul. 2019. Such compounds can also be seen in published PCT Application WO 2019/195430 A1 (Velu et al.). While such compounds are useful in the inhibition of Strep. mutans biofilm formation, continual administration to a site of interest, such as through dental rinses, may be needed. There remains a need for the incorporation of such activity into materials maintained in that environment.

### SUMMARY OF THE INVENTION

Provided herein are new materials, specifically compounds that can be incorporated into dental resins and other dental polymers that contain chemical functionality for targeted S. mutans biofilm disruption (anti-fouling). This allows “small-molecule” specificity for biological inhibition for a polymer surface. The development of biofilms on dental restorations may lead to the formation of secondary caries. One approach to disrupt/inhibit biofilm formation is to affect the formation of extracellular polymeric substances (EPS). The role of bioactive small molecules (such as G43) in targeting EPS-forming enzymes (GTFs) of bacterial species contributing to caries causing biofilm dysbiosis is investigated.

Provided herein is a compound comprising:
a) a bioactive compound;
b) a linear linking group having a first terminus and a second terminus, the first terminus and the second terminus being located at opposite ends of the linear linking group; and
c) a polymerizable end group,
wherein, the bioactive compound is bound to the first terminus of the linear linking group and the polymerizable end group being bound to the second terminus of the linear linking group.

Also provided is a method of creating a polymer capable of inhibiting the formation of Streptococcus mutans biofilms, the method comprising creating a polymeric material utilizing a compound herein.

### BRIEF DESCRIPTION OF THE DRAWINGS

FIG. 1 provides graphs representing the rate of polymerization and cell viability % achieved by test compounds G43 and G43TEG.

FIG. 2 represents S. mutans bacterial viability as measured using a metabolic activity via arenilla-reporter S. mutans(Merritt2016).

FIG. 3 Represents quantification of biofilms grown under different inoculants (50 μM) using crystal violet (CV) stain: (top) Biofilm morphology as visualized after CV stain, (chart) Colorimetrically quantified CV (Absat562 nm) to quantity the biofilm.

FIG. 4 represents quantification of biofilms grown under different inoculants (50 μM) using crystalviolet (CV) stain: (top) Biofilm morphology as visualized after CVstain, (chart) Colorimetrically quantified CV (Absat562 nm) to quantity the biofilm.

FIG. 5 represents quantification of biofilms grown using four strains of S. mutans and colorimetrically quantified CV (Absat562 nm) to quantity the biofilm. Note that AGTF denotes which Gtf enzyme(s) are not expressed by the strain.

FIG. 6 represents polysaccharide connectivity analysis of the EPS present in biofilms grown under different inoculants (50 μM) showing the ratio of different glucoselinkers. Note that α-1,3 mutans contribute to insoluble properties while α-1,6 dextrans contribute to solubility and stickiness

### DETAILED DESCRIPTION OF THE INVENTION

### Bioactive Compound

Bioactive compounds for use in the polymers described herein include those described in WO 2019/195430 A1 (Velu et al., published Oct. 10, 2019), the contents of which are incorporated herein by reference in their entirety. The bioactive compounds selectively disrupt the synthesis of extracellular matrix by S. mutans, preventing biofilm maturation by glycosyltransferase inhibition.

The bioactive compounds comprise those of Formula (I):

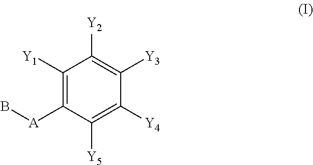

wherein:

A is —NH—, -0-, —CH₂—, —S(O)—, —S(0)₂-, —NHC(═NH)NH—, —NHC(═O)NH—, or a bond;

B is a structure selected from the group consisting of:

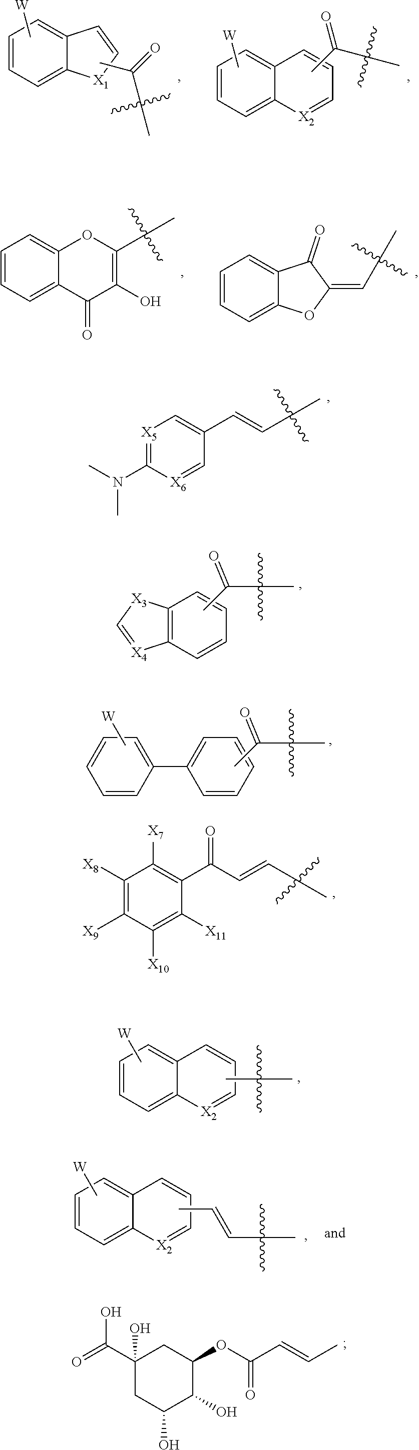

W is selected from the group consisting of —H, —N₃, -Halo (e.g., Cl, Br, F, 1), —NH₂, —NO₂, —CN, —OH, —SH, —C(═O)OH, —C(═O)H, —CH₂(Halo), —CH(Halo)₂, —C(Halo)₃, —O(C₁-C₆ alkyl) (e.g., —OCH₃), —NH(C₁-C₆ alkyl), —C(═O)NH₂, —S(C₁-C₆ alkyl), —OC(═O)(C₁-C₆ alkyl), NHC(═O)(C₁-C₆ alkyl), —C(═O)O(C₁-C₆ alkyl) (e.g., C(═O)OCH3), —C(═O)(C₁-C₆ alkyl), C(═O)NH(C₁-C₆ alkyl), —C₂-C₁₀ alkenyl, —C₂-C₁₀ alkynyl, —C₁-C₁₀ alkyl (e.g., —C₁-C₄ alkyl), and

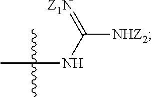

X₁, X₃, and X₄ are independently selected from the group consisting of —N—, —S-and -0-;

X₂, X5 and X₆ are independently selected from-N-or —C—;

Y₁, Y₂, Y₃, Y₄, and Ys are independently selected from the group consisting of —H, —N₃, —Halo (e.g., Cl, Br, F, I), —NO2, —CN, —OH, —SH, —C(═O)OH, —C(═O)H, —C(═O)NH₂, 4, 5 —OCH₂O—, —CH₂(Halo), —CH(Halo)₂, —C(Halo)₃, —O(C₁-C₆ alkyl) (e.g., —OCH₃), —NH(C₁-C₆ alkyl), —S(C₁-C₆ alkyl), —OC(═O)(C₁-C₆ alkyl), —NHC(═O)(C₁-C₆ alkyl), —C(═O)O(C₁-C₆ alkyl) (e.g., —C(O)OCH3), —C(═O)(C₁-C₆ alkyl), —C(═O)O(C₁-C₆ alkyl), —C(═O)NH(C₁-C₆ alkyl), CH₂—NHZ₃, —NHZ₄, —C₂-C₁₀ alkenyl, —C₂-C₁₀ alkynyl, —C₁-C₁₀ alkyl (e.g., C₁-C₄ alkyl), and

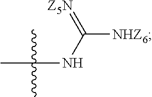

X₇, X₈, X₉, X₁₀, and X₁₁ are independently selected from the group consisting of —H, —N₃, -Halo (e.g., Cl, Br, F, I), —NO₂, —CN, —OH, —SH, —C(═O)OH, —C(═O)H, —C(═O)NH₂, CH₂(Halo), —CH(Halo)₂, —C(Halo)₃, —O(C₁-C₆ alkyl) (e.g., —OCH₃), —NH(C₁-C₆ alkyl), —CH₂—NHZ₇, —NHZs, —S(C₁-C₆ alkyl), —OC(═O)(C₁-C₆ alkyl), —NHC(═O)(C₁-C₆ alkyl), —C(═O)O(C₁-C₆ alkyl), —C(═O)(C₁-C₆ alkyl), —C(═O)NH(C₁-C₆ alkyl), —C₂-C₁₀ alkenyl, —C₂-C₁₀ alkynyl, —C₁-C₁₀ alkyl (e.g., C₁-C₄ alkyl),

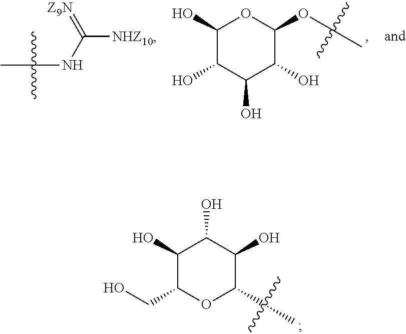

wherein Z₁, Z₂, Z₃, Z₄, Z₅, Z₆, Z₇, Z₈, Z₉, and Z₁₀ are independently selected from —H or a nitrogen protecting group; or a pharmaceutically acceptable salt thereof.

In some embodiments of the invention, the a nitrogen protecting group is selected from, but not limited to, the group of a earbobenzyloxy (Cbz) group, ap-methoxybenzyi carbonyl (Moz or MeOZ) group, a tert-butyloxycarbonyl (BOC) group, a 9-fluorenylmethyloxycarbonyl (FMOC) group, an acetyl (Ac) group, a benzoyl (Bz) group, a benzyl (Bn) group, a carbamate group, ap-methoxybenzyi (PMB) group, a 3,4-dimethoxybenzyl (DMPM) group, ap-methoxyphenyl (PMP) group, a tosyl (Ts) group, a troc (trichloroethyl chloroformate) group, and a sulfonamide (e.g., Nosyl or Nps) group.

In some embodiments of the invention, the compound of formula (I) is a compound, wherein Z₁, Z₂, Z₃, Z₄, Z₅, Z₆, Z₇, Z₈, Z₉, and Z₁₀ are independently selected from the group consisting of —H, -Boc and-Ts.

In some embodiments of the invention, the compound of formula (I) is a compound, wherein W is selected from the group consisting of —H, —N₃, -Halo, —NH₂, —NO₂, —OCH₃ and

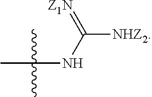

In some embodiments of the invention, the compound of formula (I) is a compound, wherein Y₁, Y₂, Y₃, Y₄, and Y₅ are independently selected from the group consisting of —H, OCH₃, —OH, —N₃, —NO₂, -Halo, —C₁-C₄ alkyl, —CH₂, NHZ₃, —NHZ₄, —C(═O)NH₂, —C(═O)OH, C(═O)OCH3, and

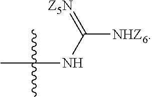

In some embodiments of the invention, the compound of formula (I) is a compound, wherein X₇, X₈, X₉, X₁₀, and X₁₁ are independently selected from the group consisting of —H, —OCH₃, —OH, —N₃, —NO₂, -Halo, C₁₋₄ alkyl, —CH₂—NHZ₇, —NHZ₈, —C(═O)NH₂, —C(═O)OH, —C(═O))CH₃,

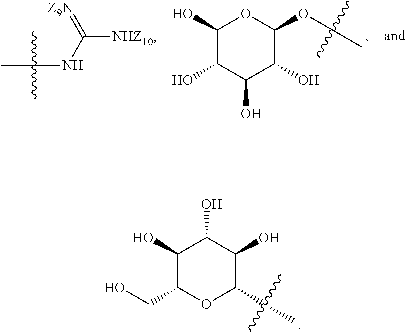

In some embodiments of the invention, the bioactive compound of formula (I) is a compound is selected from the group consisting of:

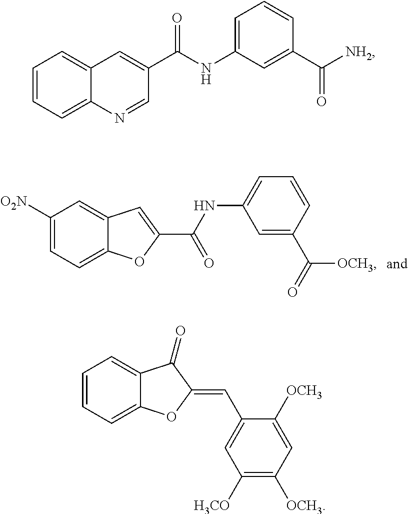 [SMILES: NC(=O)c1cccc(NC(=O)c2cnc3ccccc3c2)c1 ; COC(=O)c1cccc(NC(=O)c2cc3cc([N+](=O)[O-])ccc3o2)c1 ; COc1cc(OC)c(OC)cc1/C=C1\Oc2ccccc2C1=O]

In some embodiments of the invention, the compound of formula (I) is compound:

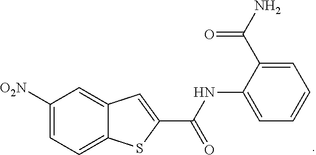 [SMILES: NC(=O)c1ccccc1NC(=O)c1cc2cc([N+](=O)[O-])ccc2s1]

In some embodiments of the invention, the compound of formula (I) is a compound, wherein the salt is a fluoride salt or a chloride salt.

In some embodiments of the invention, the compound of formula (I) is a compound, wherein the salt is a pharmaceutically acceptable salt.

In some embodiments of the invention, the compound of formula (I) is a compound selected from the group consisting of:

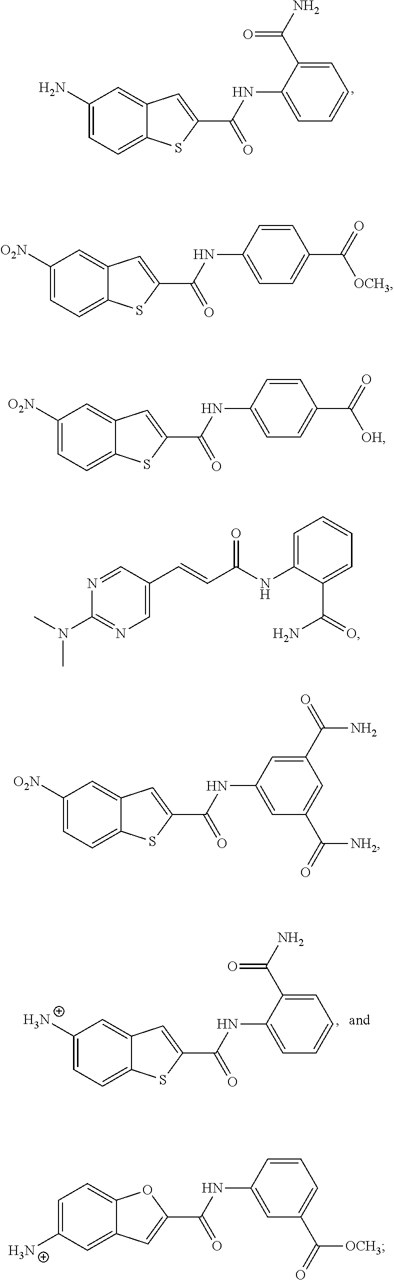

or a pharmaceutically acceptable salt thereof.

In an embodiment of the invention, the bioactive compound is a compound of formula (II):

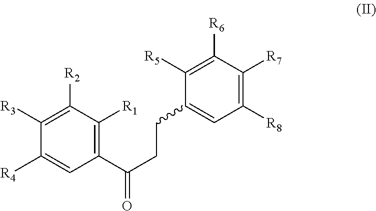

wherein:

R₁, R₂, R₃, and R₄ are independently selected from the group consisting of —H, —N₃, Halo (e.g., Cl, Br, F, I), 4, 5 —OCH₂O—, —NH₂, —NO₂, —CN, —OH, —SH, —C(═O)OH, —C(═O)H, C(═O)NH, —CH₂(Halo), —CH(Halo)₂, —C(Halo)₃, —O(C₁-C₆ alkyl) (e.g., —OCH₃), —NH(C₁-C₆ alkyl), —CHz-NHZ₁₁, —NHZ₁₂, —S(C₁-C₆ alkyl), —OC(═O)(C₁-C₆ alkyl), —NHC(═O)(C₁-C₆ alkyl), —C(═O)O(C₁-C₆ alkyl), —C(═O)(C₁-C₆ alkyl), —C(═O)NH(C₁-C₆ alkyl), —C₂-C₁₀ alkenyl, —C₂-C₁₀ alkynyl, —C₁-C₁₀ alkyl (e.g., C₁₋₄ alkyl),

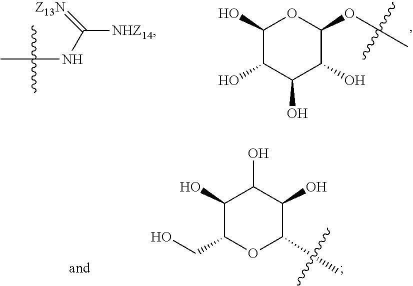

R₅, R₆, R₇, and R₈ are independently selected from the group consisting of —H, —N₃, -Halo (e.g., Cl, Br, F, I), —NO₂, —CN, —OH, —SH, —C(═O)OH, —C(═O)H, —C(═O)NH₂, CH₂(Halo), —CH(Halo)₂, —C(Halo)₃, —O(C₁-C₆ alkyl) (e.g., —OCH₃), —NH(C₁-C₆ alkyl), —S(C₁-C₆ alkyl), —OC(═O)(C₁-C₆ alkyl), —NHC(═O)(C₁-C₆ alkyl), —C(═O)O(C₁-C₆ alkyl) (e.g., C(O)OCH₃), —C(═O)(C₁-C₆ alkyl), —C(═O)O(C₁-C₆ alkyl), —C(═O)NH(C₁-C₆ alkyl), —CH₂NHZ₁₅, —NHZ₁₆—C₂-C₁₀ alkenyl, —C₂-C₁₀ alkynyl, —C₁-C₁₀ alkyl (e.g., C₁₋₄ alkyl), and

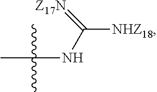

wherein Z₁₁, Z₁₂, Z₁₃, Z₁₄, Z₁₅, Z₁₆, Z₁₇ and Z₁₈ are independently selected from the group of —H or a nitrogen protecting group; or a pharmaceutically acceptable salt thereof.

In some embodiments of the invention, the a nitrogen protecting group is selected from, but not limited to, a carbobenzyloxy (Cbz) group, a p-methoxybenzyl carbonyl (Moz or MeOZ) group, a tert-butyloxycarbonyl (BOC) group, a 9-fluorenylmethyloxycarbonyl (FMOC) group, an acetyl (Ac) group, a benzoyl (Bz) group, a benzyl (Bn) group, a carbamate group, a p-methoxybenzyl (PMB) group, a 3,4-dimethoxybenzyl (DMPM) group, a p-methoxyphenyl (PMP) group, atosyl (Ts) group, a troc (trichloroethyl chloroformate) group, and a sulfonamide (e.g., Nosyl or Nps) group.

In some embodiments of the invention, the compound of formula (II) is a compound, wherein Z₁₁, Z₁₂, Z₁₃, Z₁₄, Z₁₅, Z₁₆, Z₁₇ and Z₁₈ are independently selected from the group consisting of —H, -Boc and-Ts.

In some embodiments of the invention, the compound of formula (II) is a compound wherein R₁, R₂, R₃, and R₄ are independently selected from the group consisting of —H, —OH, —OMe, —NMe₂, and 4, 5 —OCH20-.

In some embodiments of the invention, the compound of formula (II) is a compound wherein R₅, R₆, R₇, and R₈ are independently selected from the group consisting of —H, —OH, —OMe, and -Halo.

In some embodiments of the invention, the compound of formula (II) is a compound wherein at least 2 R groups selected from the group of R₅, R₆, R₇, and R₈ are —OH.

In some embodiments of the invention, the compound of formula (II) is a compound wherein R₇ is not —H.

In some embodiments of the invention, the compound of formula (II) is a compound wherein, R₇ is —OH, and one of R₅ or R₆ is —OH.

In some embodiments of the invention, the compound of formula (II) is a compound, wherein R₁ is —OH, and R₂ and R₃ are each selected independently from the group of —OH or —OMe.

In some embodiments of the invention, the compound of formula II is not a compound wherein R₇ is —OMe and

R₆ is —OH, and R₃ and R₄ are each independently selected from the group of —OMe and —OCH₂O—; or

R₃ is —OH, and R₂ and R₄ are each —NMe₂; or

R₁ and R₄ are each —OH; or

R₆ and R₈ are each —OMe, and R₃ is —OH.

In some embodiments of the invention, the bioactive compound of formula II is not a compound wherein R₅ is —OH, and

R₃ is —OH— and R₄ is OMe; or

R₁ and R₃ are each —OH; or

R₇ and R₂ are each —OH; or

R₂ is OH and R₆ and R8 are each —Cl or —H.

In some embodiments of the invention, the bioactive compound of formula II is not a compound wherein R₁ is —OH and

R₇ is —OH and R₆ is —OMe or —H; or

R₆ or R₃ is OH; or

R₅, R₇, and R₈ are —OH.

In some embodiments of the invention, the bioactive compound of formula (II) is selected from the group of:

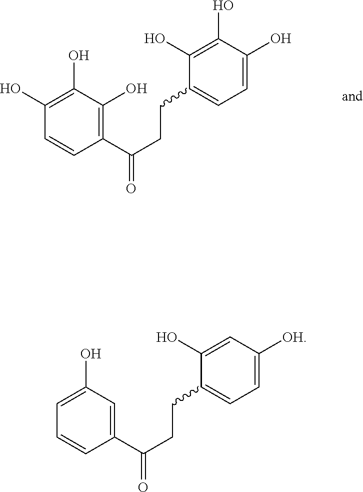 [SMILES: O=C(CCc1ccc(O)c(O)c1O)c1ccc(O)c(O)c1O ; O=C(CCc1ccc(O)cc1O)c1cccc(O)c1]

In other embodiments of the invention, the bioactive compound of formula (II) is selected from the group of:

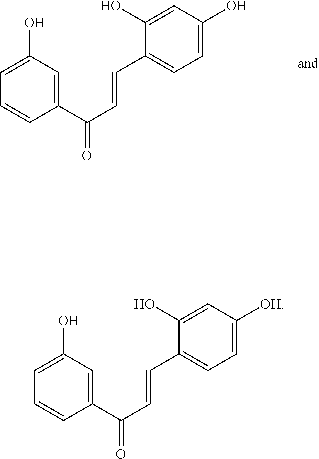 [SMILES: O=C(/C=C/c1ccc(O)cc1O)c1cccc(O)c1 ; O=C(/C=C/c1ccc(O)cc1O)c1cccc(O)c1]

In other embodiments, the bioactive compound is a compound of Formula IA or IB:

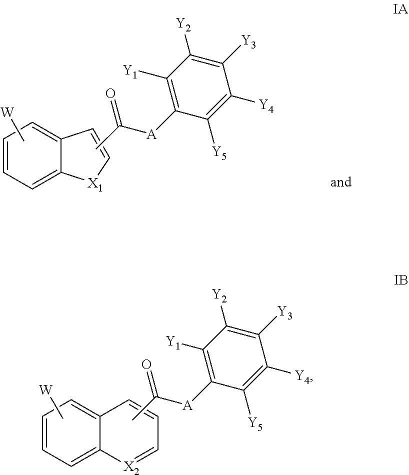

In some embodiments, the bioactive compound of Formula IA or Formula IB is selected from the group of the following, or a pharmaceutically acceptable salt thereof:

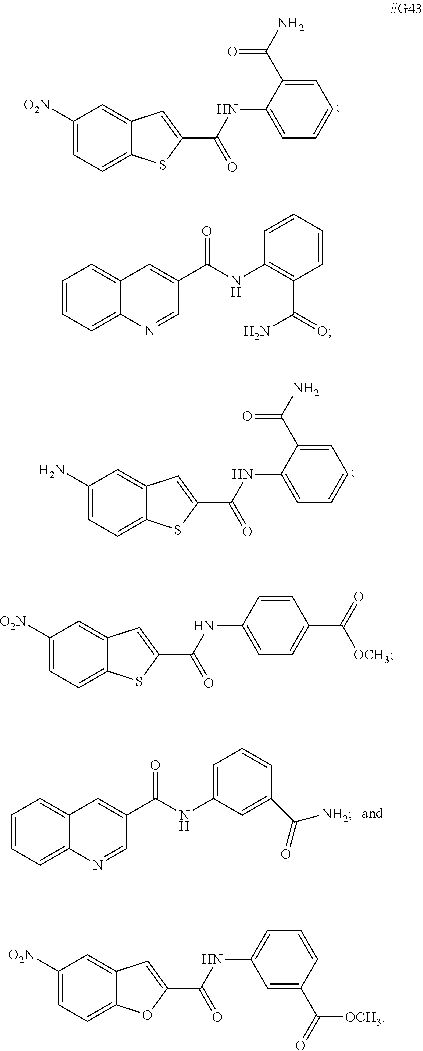 [SMILES: NC(=O)c1ccccc1NC(=O)c1cc2cc([N+](=O)[O-])ccc2s1 ; NC(=O)c1ccccc1NC(=O)c1cnc2ccccc2c1 ; NC(=O)c1ccccc1NC(=O)c1cc2cc(N)ccc2s1 ; COC(=O)c1ccc(NC(=O)c2cc3cc([N+](=O)[O-])ccc3s2)cc1 ; NC(=O)c1cccc(NC(=O)c2cnc3ccccc3c2)c1 ; COC(=O)c1cccc(NC(=O)c2cc3cc([N+](=O)[O-])ccc3o2)c1]

In other embodiments, the bioactive compound is selected from the group below, or a pharmaceutically acceptable salt thereof:

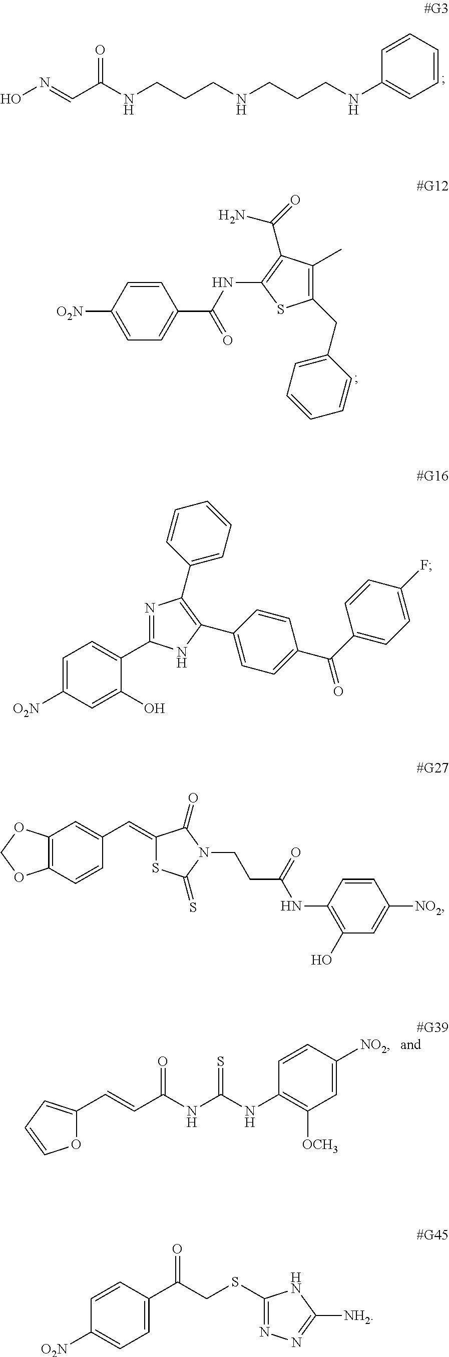 [SMILES: O=C(/C=N/O)NCCCNCCCNc1ccccc1 ; Cc1c(Cc2ccccc2)sc(NC(=O)c2ccc([N+](=O)[O-])cc2)c1C(N)=O ; O=C(c1ccc(F)cc1)c1ccc(-c2[nH]c(-c3ccc([N+](=O)[O-])cc3O)nc2-c2ccccc2)cc1 ; O=C(CCN1C(=O)/C(=C/c2ccc3c(c2)OCO3)SC1=S)Nc1ccc([N+](=O)[O-])cc1O ; COc1cc([N+](=O)[O-])ccc1NC(=S)NC(=O)/C=C/c1ccco1 ; Nc1nnc(SCC(=O)c2ccc([N+](=O)[O-])cc2)[nH]1]

In still other embodiments, the bioactive compound is selected from the group below, or a pharmaceutically acceptable salt thereof:

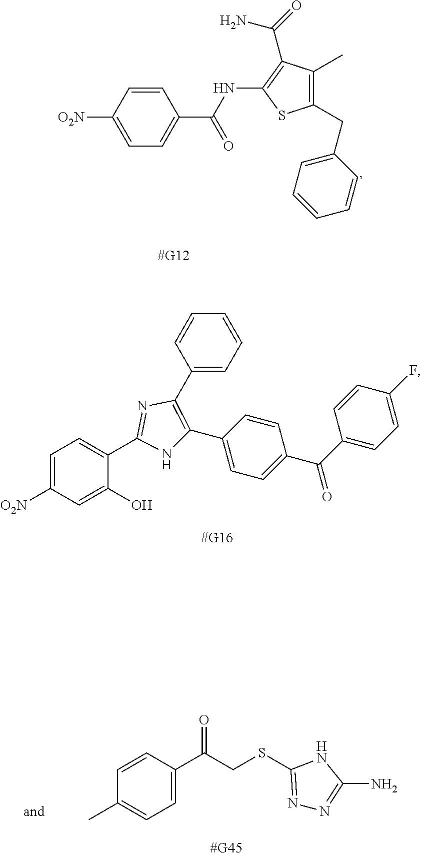 [SMILES: Cc1c(Cc2ccccc2)sc(NC(=O)c2ccc([N+](=O)[O-])cc2)c1C(N)=O ; O=C(c1ccc(F)cc1)c1ccc(-c2[nH]c(-c3ccc([N+](=O)[O-])cc3O)nc2-c2ccccc2)cc1 ; Cc1ccc(C(=O)CSc2nnc(N)[nH]2)cc1]

The bioactive compounds described above may be prepared by methods known in the art, including those described in WO 2019/195430 A1.

### Linear Linking Groups

Linking groups in the compounds herein may be selected from each of the following, wherein n is an integer selected from the group of 1 to 20. In some embodiments, n is an integer selected from the group of from 2 to 15. In other embodiments, n is an integer selected from the group of 2 to 12. In further embodiments, n is an integer selected from 2 to 10. In additional embodiments, n is an integer of from 2 to 8. In further embodiments, n is an integer from 2 to 6.

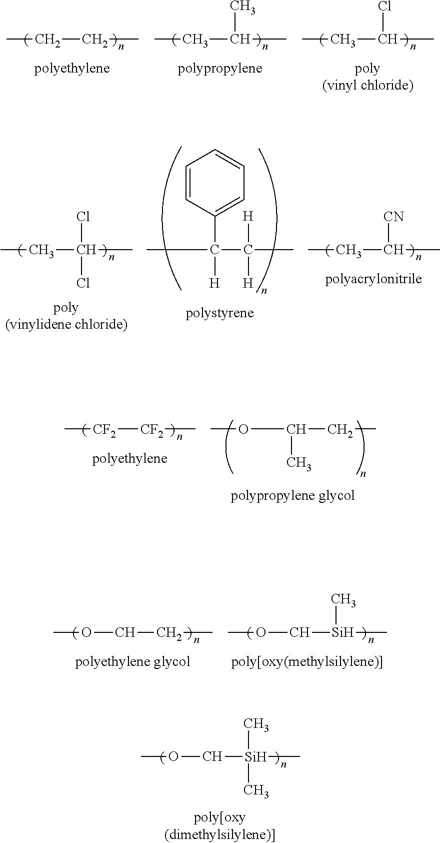

### Polymerizable End Groups

Polymerizable functional groups as end groups may include those having a carboxylic acid or at least one ethylenically unsaturated group. Examples include endgroups formed from a terminal group of acrylic acid (including mono-, di-, and tri-acrylates), maleic acid, maleic anhydride, itaconic acid, methacrylic acid, 4-methacryloxyethyltrimellitic anhydride, (meth)acrylated homopolymer or copolymer of an α,β-unsaturated carboxylic acid {e.g. (meth)acrylated poly(acrylic acid), (meth)acrylated poly(acrylic acid-maleic acid) copolymer and (meth)acrylated poly(acrylic-maleic acid-itaconic acid) copolymer}, any addition product of mono-/dianhydride compound with a hydroxyalkylmethacrylate compound (e.g. the addition product of pyromellitic acid anhydride and glycerol dimethacrylate, the addition product of 3,3′,4,4′ benzophenonetetra carboxylic dianhydride and hydroxyethyl methacrylate, the addition product of phthalic anhydride and hydroxyethyl methacrylate, and the addition product of maleic anhydride and glycerol dimethacrylate), methyl (meth)acrylate, ethyl (meth)acrylate, propyl (meth)acrylate, butyl (meth)acrylate, hexyl (meth)acrylate, octyl (meth)acrylate, lauryl (meth)acrylate, decyl (meth)acrylate, tridecyl (meth)acrylate, 2-ethoxyethyl (meth)acrylate, 2′-ethoxy-2-ethoxyethyl (meth)aery late, ethyleneglycol di(meth)acrylate, diethyleneglycol di(meth)acrylate, polyethyleneglycol mono-(meth)acrylate, polypropyleneglycol mono-(meth)acrylate, polytetramethyleneglycol mono(meth)acrylate, hexanediol di(meth)acrylate, tetrahydrofurfuryl (meth)acrylate, N,N′-methylenebis(acrylamide), N,N′ethylenebis(acrylamide), and N,N′-butylenebis(acrylamide), or any mixture thereof.

Non-limiting examples of functional and polymerizable end groups include those of the formulas below.

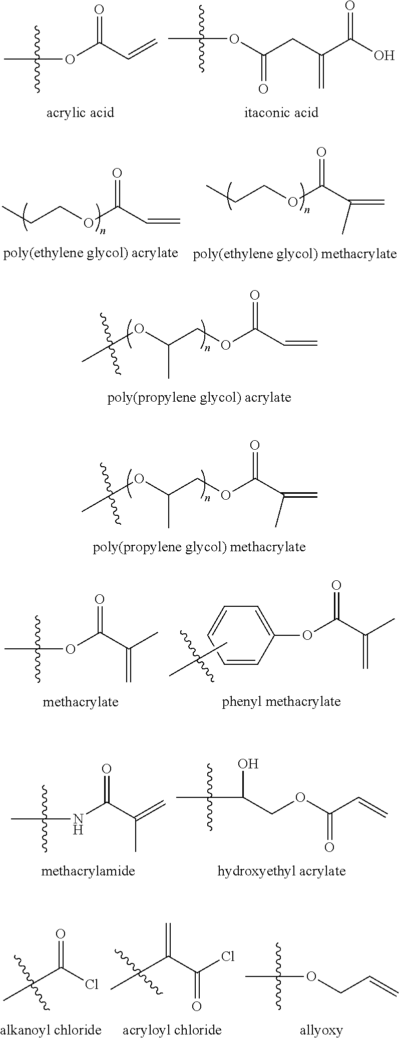

Polyacrylates, such as the di-acrylate and tri-acrylate below, may also be used.

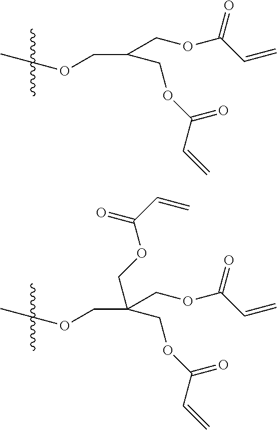

Carboxylic acid end groups may also be in the form of esters and substituted esters, such as hydroxyalkyl esters, as shown below for a terephthalic acid end group, along with its methyl ester and hydroxyethyl ester.

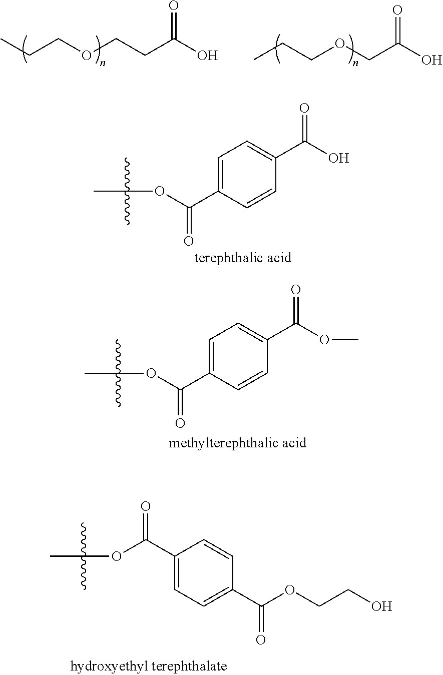

It is understood that included in the list of acid containing compounds above are esters thereof, including alkyl esters of from 1 to 10 carbon atoms and benzyl esters.

The compounds described herein may be incorporated with a full range of polymers used in dental applications, including polymers comprising rubber, acrylic resins, vinyl acrylics, epoxy resins, polyethers, polysulphides, silicones, polycarbonates, polyacrylics, polystyrenes, polyethylenes, polyamides, polyetheretherketone, polytetrafluoroethylene, and poly vinyl acetate.

These include dental polymers comprising monomers including bisphenol-A glycidylmethacrylate (bis-GMA), triethylene glycol dimethacrylate (TEGDMA), 4-methacryloxyethyl trimellitic acid (4-MET), 10-methacryloyloxydecyl dihydrogen phosphate monomer (10-MDP), 2-methacryloxyethyl phenyl hydrogen phosphate (Phenyl-P), urethane dimethacrylate (UDMA), polymethyl methacrylate (PMMA), 2-hydroxyethyl methacrylate (HEMA), isobornyl methacrylate, methyl methacrylate, 3-(trimethoxysilyl) propyl methacrylate, trimethylsilyl methacrylate, 3-tris(trimethylsilyloxy) silyl) propyl methacrylate, 2-(trimethylsilyloxy) ethyl methacrylate, bis (2-(methacryloyloxy)ethyl) phosphate, ethylene glycol methacrylate phosphate, polyethylene glycol (PEG), polydimethyolsiloxane, polyurethane, polylactic acid (PLLA), polypyrrole (PPy), hexamethyldisilazane (HMDC), N-isopropylacrylamide, N-tert-butylacrylamide, and hydrogels.

The polymers with which the present compounds may be used include those comprising crosslinking agents and resins such as 2-hydroxy-5-N-methacrylamidobenzoic acid, ethylene glycol dimethacrylate (EGDM), biphenyl dimethacrylate (BisDMA), 1,6 hexanediol dimethacrylate (HDMA), tetraethylene glycol dimethacrylate, pyromellitic dianhydrate dimethacrylate, 1,10-decamethylene glycol dimethacrylate, bisphenol A glycerolate diacrylate, glycerol 1,3-dimethacrylate (GDMA), polyethylene glycol diacrylates, dipenaerythritol penta-hexa-acrylate, ethoxylated bisphenol methacrylate (EBPADMA), bisphenol A ethoxylate diacrylate, and trimethylolpropane trimethacrylate (TMPTMA).

The compounds herein may also be incorporated into epoxy resins and related silorane (such as those offered under the FILTEK™ tradename by 3M) materials.

The compounds herein may be incorporated into dentin bonding agents, cavity fillings, resin and glass-ionomer cements, splinting materials, pit and fissure sealants, root canal sealants, veneers, brackets, bonding resins and cements, and spacers. The compounds herein may also be used in prosthodontics materials, including denture bases and teeth, core buildup materials, temporary restorative materials and products, cementing/luting materials, films, and maxillofacial prostheses.

In some embodiments, the compounds herein may be incorporated into a mix of desired monomers prior to polymerization. In some embodiments, a mix of desired monomers may be formed and one or more compounds herein may be applied to the surface of the mixture prior to polymerization.

As used herein, the term “Pharmaceutically acceptable salts” includes, for example, salts with inorganic acids and salts with an organic acid. Examples of salts may include hydrochloride, phosphate, diphosphate, hydrobromide, sulfate, sulfinate, nitrate, malate, maleate, fumarate, tartrate, succinate, citrate, acetate, lactate, methanesulfonate (mesylate), benzenesuflonate (besylate), p-toluenesulfonate (tosylate), 2-hydroxyethylsulfonate, benzoate, salicylate, stearate, and alkanoate (such as acetate, HOOC—(CH₂)ₙ—COOH where n is 0-4). In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts.

### Synthesis

Compounds of the present invention can be prepared by methods known in the art, represented by the non-limiting scheme below directed to a polymeric compound using bioactive agent N-(2-carbamoylphenyl)-5-nitrobenzo[b]thiophene-2-carboxamide (also known as #g43).

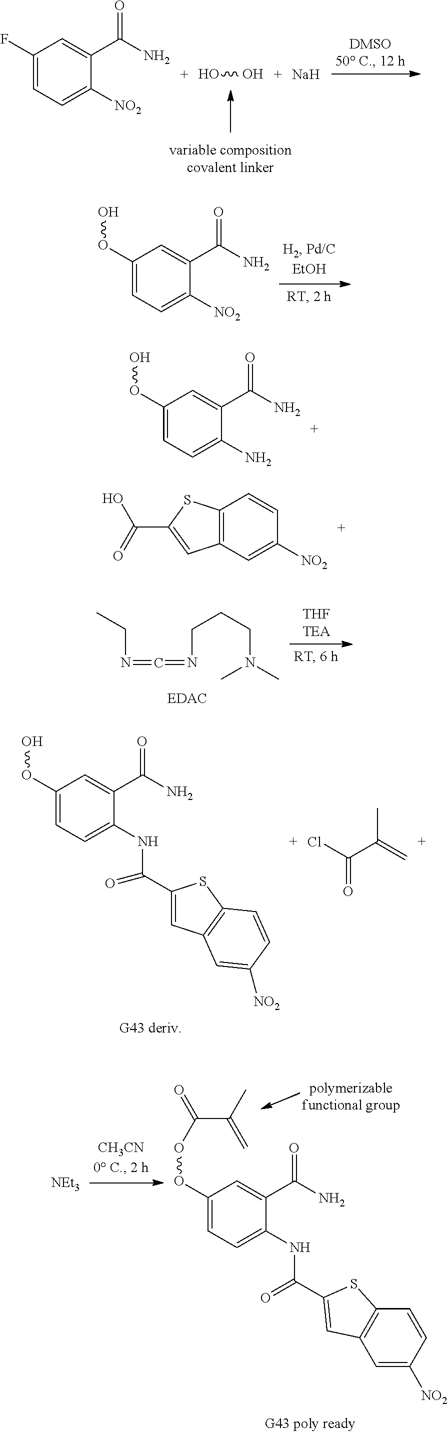

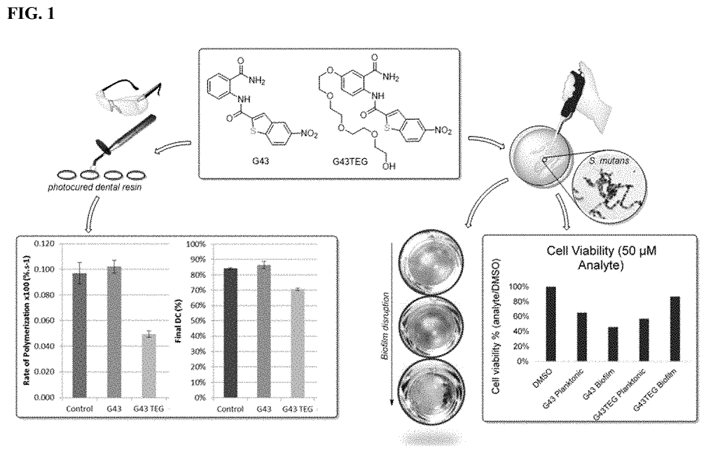

Methods: G43, and its non-binding counterpart, G43TEG (N-(2-carbamoyl-4-(2-(2-(2-(2-hydroxyethoxy)ethoxy)ethoxy)ethoxy)phenyl)-5-nitrobenzo[b]thiophene-2-carboxamide), were synthesized and minimum inhibitory concentration (MIC) against S. mutans was established. The compounds were serially diluted in DMSO to 400, 200, 100, 50, 25 and 12.5 then added to S. mutans grown in TH medium plus sucrose. Cultures were incubated for 96 h at 37° C. aerobically. Supernatants with planktonic cells and biofilms were assayed separately for metabolic activity using S. mutans carrying a renilla luciferase reporter. G43 or G43TEG were added at 4 wt % into 50:50 BisGMA/TEGDMA and 70 wt % filler. 0.2 wt % BAPO, 0.05 wt % BHT were used as the photoinitiator and inhibitor, respectively. Polymerization kinetics were assessed for 5 min by near-IR in discs (4 mm by 10 mm diameter), cured with a LED light source (700 mW/cm2, 30 s). Discs were then immersed in deionized H₂O for 7 days, with extracted monomers collected every 24 h. Results were analyzed with one-way ANOVA/Tukey's test (α=0.05).
Results: The MIC was determined to be 25-50 μM for both G43 and G43TEG with a significant reduction of viable biofilm cells (FIG. 1). Polymerizations of dental resins were relatively unaffected by 4% G43 or G43TEG (FIG. 1). Analysis of leachates showed no detectible G43 or G43TEG (<11 μM).
Conclusions: This study demonstrated that bioactive molecules in the G43 family affect S. mutans biofilm viability (25-50 Further, G43 and G43TEG did not significantly affect polymerization kinetics.

Eight G43 derivatives (G43, G43TEG, G43-TEG-MMA, G43-A2-OMe, G43-A-NH₂, G43B-Furan, G43-B1-OMe, G43-C3-OMe, and G16) were synthesized de novo screened against S. mutans for biofilm inhibition. The compounds were dissolved in DMSO and serially diluted in H₂O at 50 μM, then added to S. mutans cultures (TH medium with sucrose) & incubated for 96 h at 37° C. aerobically. Biofilms were assayed for bacterial viability (metabolic activity via a renilla-reporter S. mutans—Merritt2016). Biofilms were stained with crystal violet (CV), washed 3× with DI H2O, and then the CV was volumetrically extracted to colorimetrically determine the quantity of biofilm. Next, biofilms were washed 3× with DI H2O, then sterilized with EtOH, azeotropically dried. Insoluble biofilm was chemically digested by 1) methylation, 2) acid depolymerization, 3) reduction, and 4) acylation, resulting in methylated alditol acetates for polysaccharide connectivity determination by GC/MS. Results were analyzed with one-way ANOVA/Tukey's test (α=0.05).

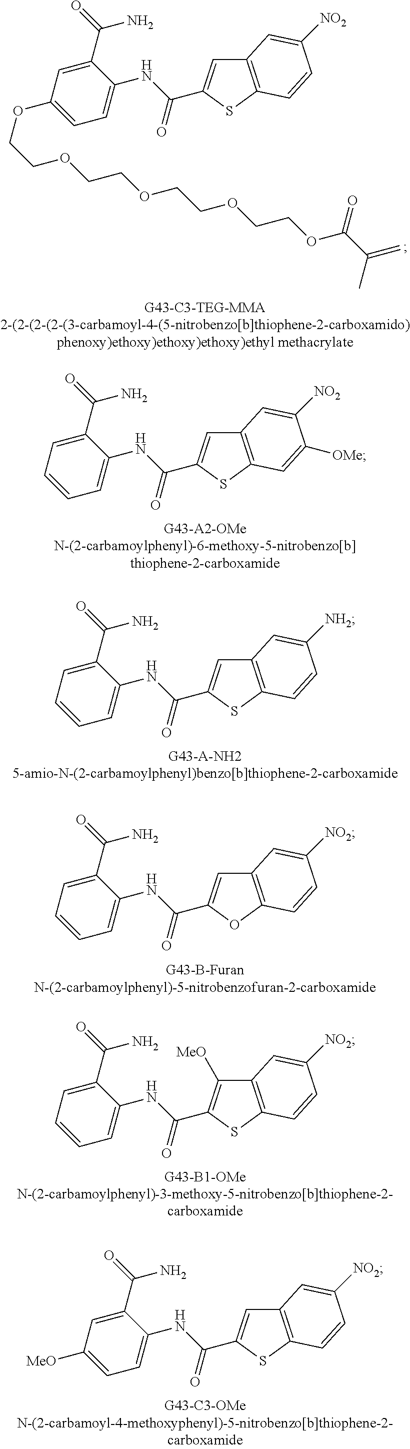 [SMILES: C=C(C)C(=O)OCCOCCOCCOCCOc1ccc(NC(=O)c2cc3cc([N+](=O)[O-])ccc3s2)c(C(N)=O)c1 ; COc1cc2sc(C(=O)Nc3ccccc3C(N)=O)cc2cc1[N+](=O)[O-] ; NC(=O)c1ccccc1NC(=O)c1cc2cc(N)ccc2s1 ; NC(=O)c1ccccc1NC(=O)c1cc2cc([N+](=O)[O-])ccc2o1 ; COc1c(C(=O)Nc2ccccc2C(N)=O)sc2ccc([N+](=O)[O-])cc12 ; COc1ccc(NC(=O)c2cc3cc([N+](=O)[O-])ccc3s2)c(C(N)=O)c1]

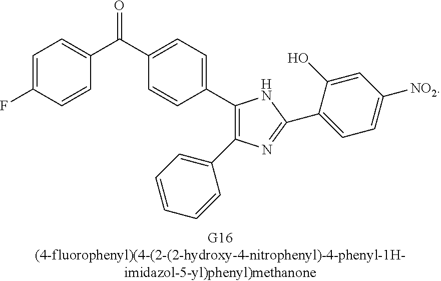 [SMILES: O=C(c1ccc(F)cc1)c1ccc(-c2[nH]c(-c3ccc([N+](=O)[O-])cc3O)nc2-c2ccccc2)cc1]

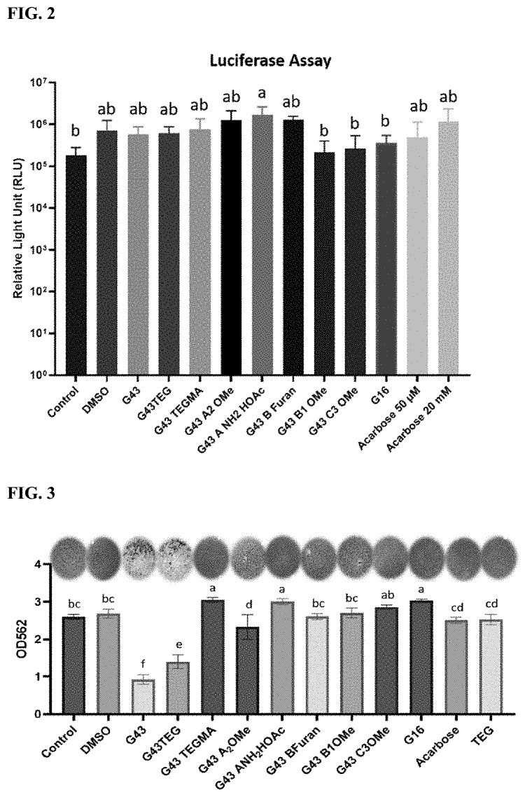

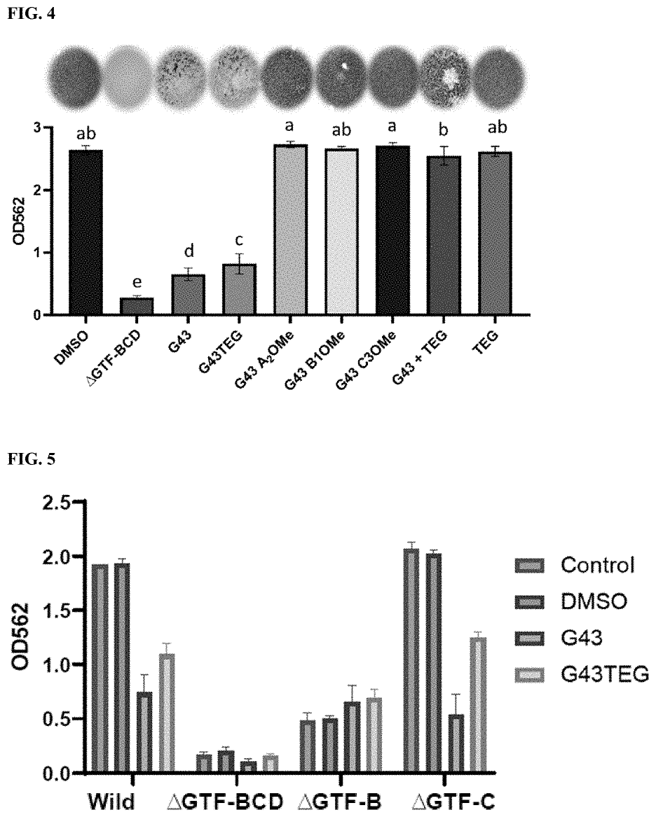

The G43 family did not significantly reduce the viability of S. mutans at 50 μM (FIG. 2).

G43 and G43TEG significantly reduced biofilm production at 50 μM (FIGS. 3 & 4).

G43 and G43TEG likely affect more than just GtfC as seen by the biofilm reduction efficacy in GtfC mutants (FIG. 5).

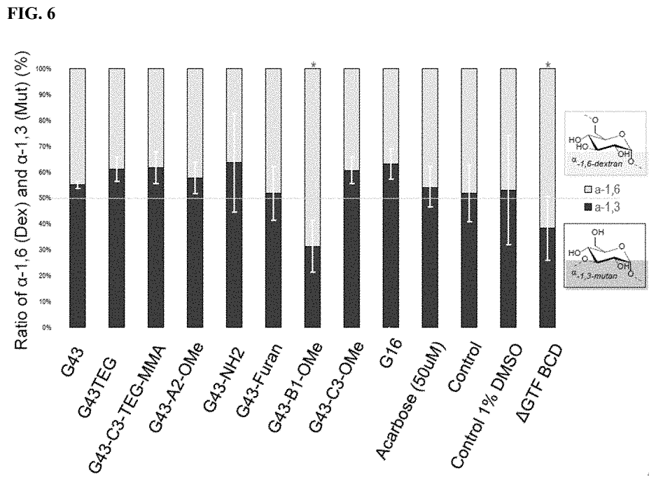

Only G43-B1-OMe significantly changed the composition of the biofilm as measured by polysaccharide connectivity (FIG. 6), even though total biofilm production was not suppressed.

What is claimed:

1. A compound comprising:
d) a bioactive compound;
e) a linear linking group having a first terminus and a second terminus, the first terminus and the second terminus being located at opposite ends of the linear linking group; and
f) a polymerizable end group;
wherein, the bioactive compound is bound to the first terminus of the linear linking group and the polymerizable end group being bound to the second terminus of the linear linking group; and
further wherein the bioactive compound is a compound of Formula (I): 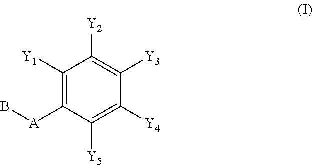
A is —NH—, -0-, —CH₂—, —S(O)—, —S(O)₂—, —NHC(═NH)NH—, —NHC(═O)NH—, or a bond;
B is a structure selected from the group consisting of: 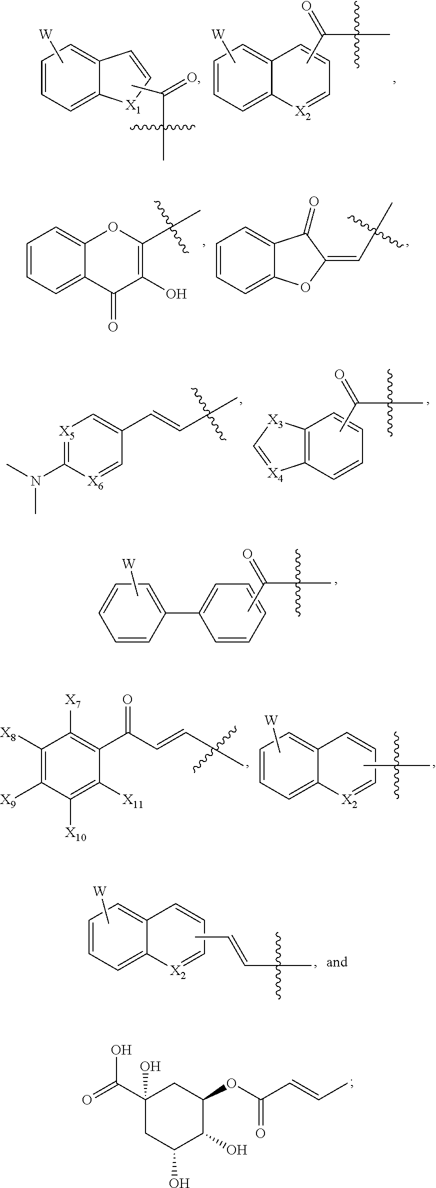
W is selected from the group consisting of —H, —N₃, -Halo (e.g., Cl, Br, F, 1), —NH₂, —NO₂, —CN, —OH, —SH, —C(═O)OH, —C(═O)H, —CH₂(Halo), —CH(Halo)₂, —C(Halo)₃, —O(C₁-C₆ alkyl) (e.g., —OCH₃), —NH(C₁-C₆ alkyl), —C(═O)NH₂, —S(C₁-C₆ alkyl), —OC(═O)(C₁-C₆ alkyl), NHC(═O)(C₁-C₆ alkyl), —C(═O)O(C₁-C₆ alkyl) (e.g., C(═O)OCH3), —C(═O)(C₁-C₆ alkyl), C(═O)NH(C₁-C₆ alkyl), —C₂-C₁₀ alkenyl, —C₂-C₁₀ alkynyl, —C₁-C₁₀ alkyl (e.g., C₁-C₄ alkyl), and 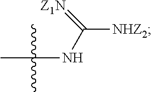
X₁, X₃, and X₄ are independently selected from the group consisting of —N—, —S-and -0-;
X₂, X5 and X₆ are independently selected from-N-or —C—;
Y₁, Y₂, Y₃, Y₄, and Ys are independently selected from the group consisting of —H, —N₃, -Halo (e.g., Cl, Br, F, I), —NO₂, —CN, —OH, —SH, —C(═O)OH, —C(═O)H, —C(═O)NH₂, 4, 5 —OCH₂O—, —CH₂(Halo), —CH(Halo)₂, —C(Halo)₃, —O(C₁-C₆ alkyl) (e.g., —OCH₃), —NH(C₁-C₆ alkyl), —S(C₁-C₆ alkyl), —OC(═O)(C₁-C₆ alkyl), —NHC(═O)(C₁-C₆ alkyl), —C(═O)O(C₁-C₆ alkyl) (e.g., —C(O)OCH3), —C(═O)(C₁-C₆ alkyl), —C(═O)O(C₁-C₆ alkyl), —C(═O)NH(C₁-C₆ alkyl), CH₂—NHZ₃, —NHZ₄, —C₂-C₁₀ alkenyl, —C₂-C₁₀ alkynyl, —C₁-C₁₀ alkyl (e.g., C₁-C₄ alkyl), and 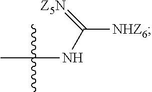
X₇, X₈, X₉, X₁₀, and X₁₁ are independently selected from the group consisting of —H, —X₃, -Halo (e.g., Cl, Br, F, I), —NO₂, —CN, —OH, —SH, —C(═O)OH, —C(═O)H, —C(═O)NH₂, CH₂(Halo), —CH(Halo)₂, —C(Halo)₃, —O(C₁-C₆ alkyl) (e.g., —OCH₃), —NH(C₁-C₆ alkyl), —CH₂—NHZ₇, —NHZs, —S(C₁-C₆ alkyl), —OC(═O)(C₁-C₆ alkyl), —NHC(═O)(C₁-C₆ alkyl), —C(═O)O(C₁-C₆ alkyl), —C(═O)(C₁-C₆ alkyl), —C(═O)NH(C₁-C₆ alkyl), —C₂-C₁₀ alkenyl, —C₂-C₁₀ alkynyl, —C₁-C₁₀ alkyl (e.g., C₁-C₄ alkyl), 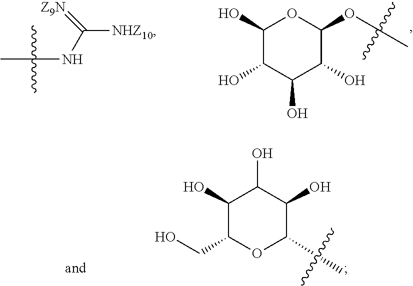
wherein Z₁, Z₂, Z₃, Z₄, Z₅, Z₆, Z₇, Z₈, Z₉, and Z₁₀ are independently selected from —H or a nitrogen protecting group; and
the linear linking group having a first terminus and a second terminus is selected from the group of: 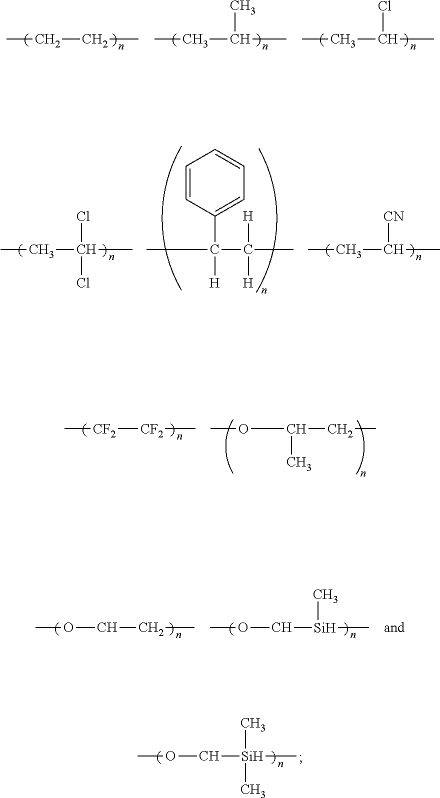
wherein n in each instance is an integer selected from 1, 2, 3, 4, 5, 6, 7, 8, 9, 10, 11, 12, 13, 14, 15, 16, 17, 18, 19, and 20; and
the polymerizable end group is selected from the group of: 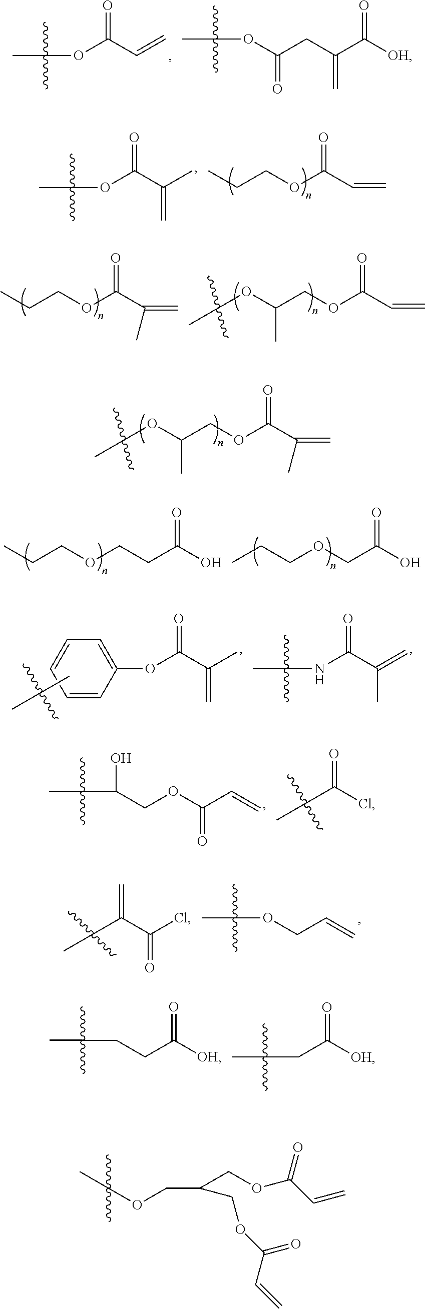 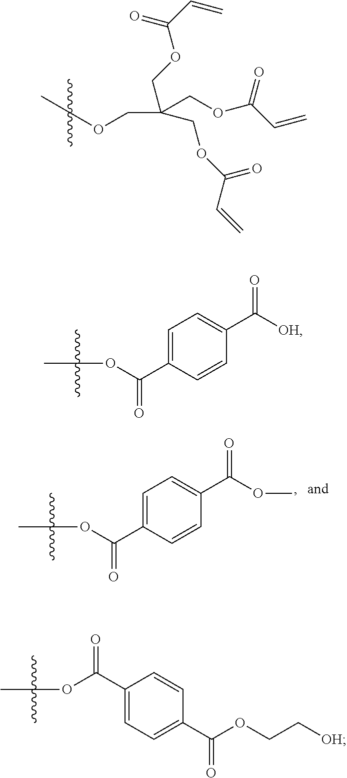
wherein n is an integer independently selected in each instance from the group of 1, 2, 3, 4, 5, 6, 7, 8, 9, 10, 11, 12, 13, 14, 15, 16, 17, 18, 19, and 20; or a pharmaceutically acceptable salt thereof.

2. The compound of claim 1, wherein Z₁, Z₂, Z₃, Z₄, Z₅, Z₆, Z₇, Z₈, Z₉, and Z₁₀ are independently selected from the group consisting of —H, -Boc and-Ts.

3. The compound of claim 1, wherein W is selected from the group consisting of —H, —N₃, -Halo, —NH₂, —NO₂, —OCH₃ and

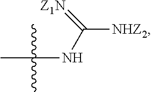

4. The compound of claim 1, wherein Y₁, Y₂, Y₃, Y₄, and Y₅ are independently selected from the group consisting of —H, OCH₃, —OH, —N₃, —NO₂, -Halo, —C₁-C₄ alkyl, —CH₂, —NHZ₃, —NHZ₄, —C(═O)NH₂, —C(═O)OH, C(═O)OCH₃, and

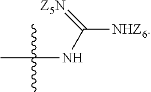

5. The compound of claim 1, wherein, X₇, X₈, X₉, X₁₀, and Xu are independently selected from the group consisting of —H, —OCH₃, —OH, —N₃, —NO₂, -Halo, C₁₋₄ alkyl, —CH₂—NHZ₇, —NHZ₈, —C(═O)NH₂, —C(═O)OH, —C(═O))CH₃,

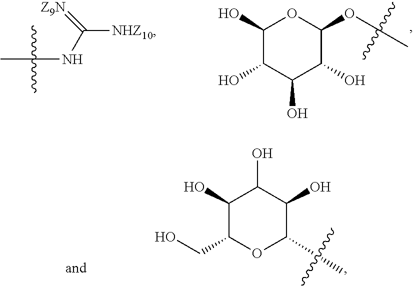

6. The compound of claim 1, wherein the bioactive compound is selected from the group of:

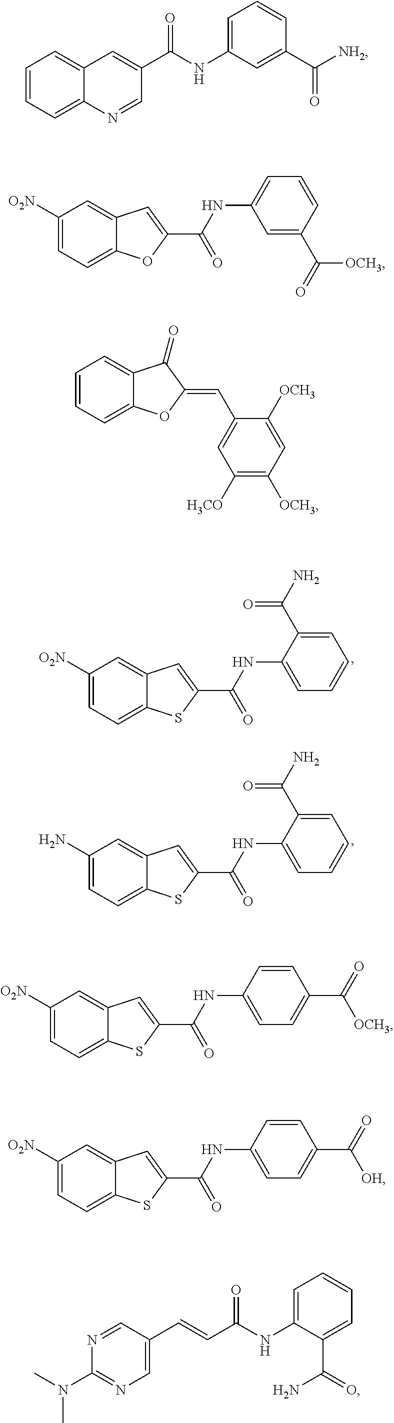

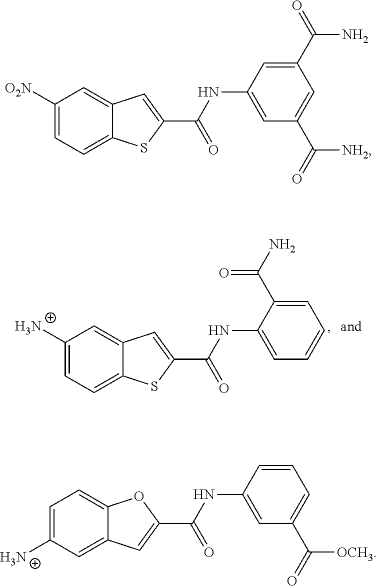 [SMILES: NC(=O)c1cc(NC(=O)c2cc3cc([N+](=O)[O-])ccc3s2)cc(C(N)=O)c1 ; NC(=O)c1ccccc1NC(=O)c1cc2cc([NH3+])ccc2s1 ; COC(=O)c1cccc(NC(=O)c2cc3cc([NH3+])ccc3o2)c1]

7. The compound of claim 1, wherein the bioactive compound has the formula (II): 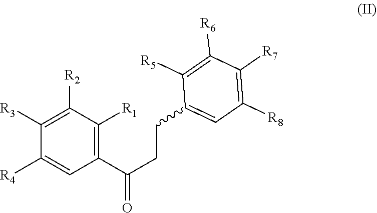
wherein:
R₁, R₂, R₃, and R₄ are independently selected from the group consisting of —H, —N₃, Halo (e.g., Cl, Br, F, I), 4, 5 —OCH₂O—, —NH₂, —NO₂, —CN, —OH, —SH, —C(═O)OH, —C(═O)H, C(═O)NH, —CH₂(Halo), —CH(Halo)₂, —C(Halo)₃, —O(C₁-C₆ alkyl) (e.g., —OCH₃), —NH(C₁-C₆ alkyl), —CHz-NHZ₁₁, —NHZ₁₂, —S(C₁-C₆ alkyl), —OC(═O)(C₁-C₆ alkyl), —NHC(═O)(C₁-C₆ alkyl), —C(═O)O(C₁-C₆ alkyl), —C(═O)(C₁-C₆ alkyl), —C(═O)NH(C₁-C₆ alkyl), —C₂-C₁₀ alkenyl, —C₂-C₁₀ alkynyl, —C₁-C₁₀ alkyl (e.g., C₁₋₄ alkyl), 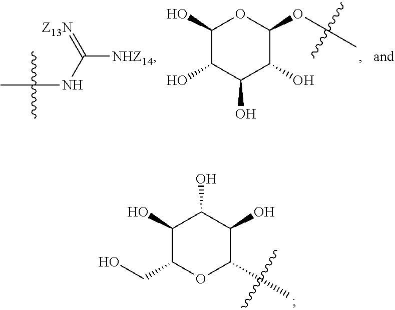
R₅, R₆, R₇, and R₈ are independently selected from the group consisting of —H, —N₃, -Halo (e.g., Cl, Br, F, I), —NO₂, —CN, —OH, —SH, —C(═O)OH, —C(═O)H, —C(═O)NH₂, CH₂(Halo), —CH(Halo)₂, —C(Halo)₃, —O(C₁-C₆ alkyl) (e.g., —OCH₃), —NH(C₁-C₆ alkyl), —S(C₁-C₆ alkyl), —OC(═O)(C₁-C₆ alkyl), —NHC(═O)(C₁-C₆ alkyl), —C(═O)O(C₁-C₆ alkyl) (e.g., C(O)OCH₃), —C(═O)(C₁-C₆ alkyl), —C(═O)O(C₁-C₆ alkyl), —C(═O)NH(C₁-C₆ alkyl), —CH₂NHZ₁₅, —NHZ₁₆—C₂-C₁₀ alkenyl, —C₂-C₁₀ alkynyl, —C₁-C₁₀ alkyl (e.g., C₁₋₄ alkyl), and 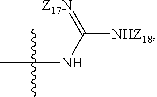
wherein Z₁₁, Z₁₂, Z₁₃, Z₁₄, Z₁₅, Z₁₆, Z₁₇ and Z₁₈ are independently selected from the group of —H or a nitrogen protecting group; or a pharmaceutically acceptable salt thereof.

8. The compound of claim 7, wherein the compound of Formula (II) is selected from the group of compounds below, or a pharmaceutically acceptable salt thereof:

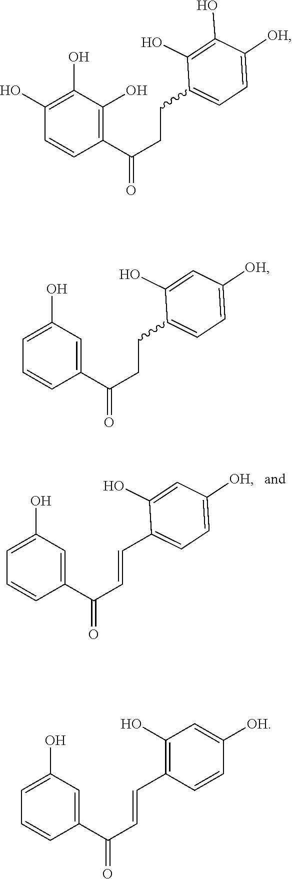 [SMILES: O=C(CCc1ccc(O)c(O)c1O)c1ccc(O)c(O)c1O ; O=C(CCc1ccc(O)cc1O)c1cccc(O)c1 ; O=C(/C=C/c1ccc(O)cc1O)c1cccc(O)c1 ; "and" ; O=C(/C=C/c1ccc(O)cc1O)c1cccc(O)c1]

9. The compound of claim 1, wherein the bioactive compound is a compound of Formula IA, or a pharmaceutically acceptable salt thereof: 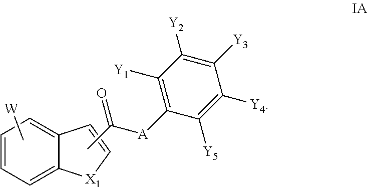
wherein W, X₁, A, Y₁, Y₂, Y₃, Y₄, and Y₅ are as defined in claim 1.

10. The compound of claim 9, wherein the bioactive compound is selected from the group of compounds below, or a pharmaceutically acceptable salt thereof:

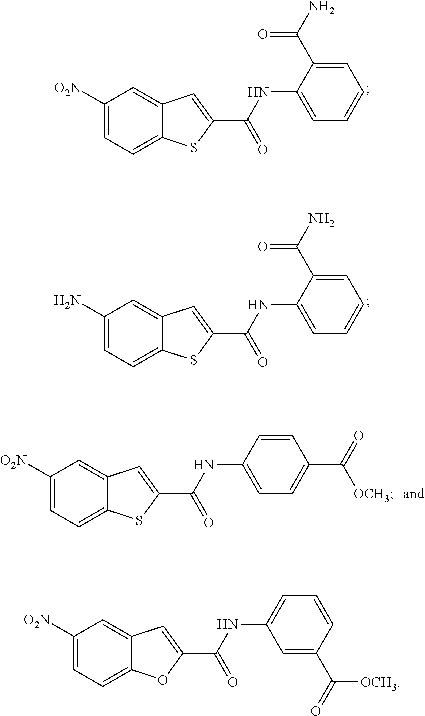 [SMILES: NC(=O)c1ccccc1NC(=O)c1cc2cc([N+](=O)[O-])ccc2s1 ; NC(=O)c1ccccc1NC(=O)c1cc2cc(N)ccc2s1 ; COC(=O)c1ccc(NC(=O)c2cc3cc([N+](=O)[O-])ccc3s2)cc1 ; COC(=O)c1cccc(NC(=O)c2cc3cc([N+](=O)[O-])ccc3o2)c1]

11. The compound of claim 1, wherein the bioactive compound is a compound of Formula IB, or a pharmaceutically acceptable salt thereof: 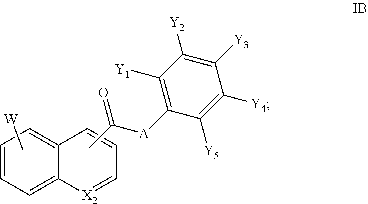
wherein W, X₂, A, Y₁, Y₂, Y₃, Y₄, and Y₅ are as defined in claim 1.

12. The compound of claim 11, wherein the bioactive compound is selected from the group of compounds below, or a pharmaceutically acceptable salt thereof:

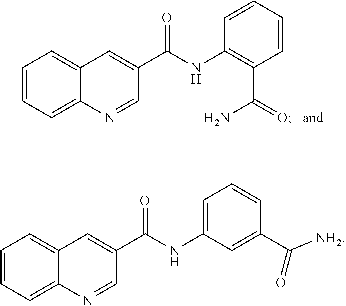 [SMILES: NC(=O)c1ccccc1NC(=O)c1cnc2ccccc2c1 ; "and" ; NC(=O)c1cccc(NC(=O)c2cnc3ccccc3c2)c1]

13. The compound of claim 1, wherein the bioactive compound is selected from the group of compounds below, or a pharmaceutically acceptable salt thereof:

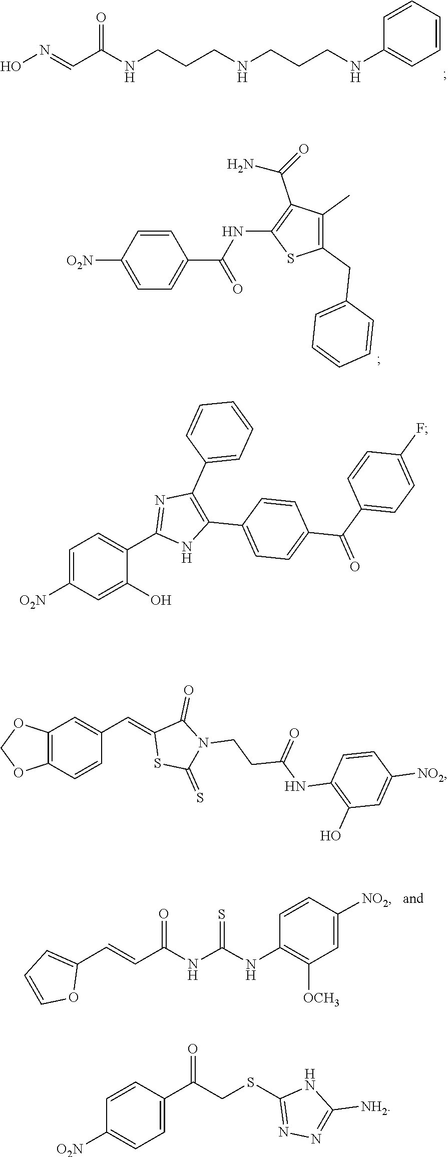

14. The compound of claim 1, wherein the bioactive compound is selected from the group of compounds below, or a pharmaceutically acceptable salt thereof.

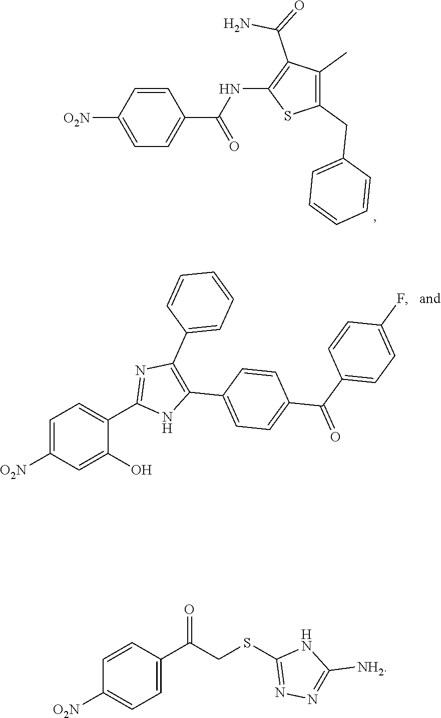 [SMILES: Cc1c(Cc2ccccc2)sc(NC(=O)c2ccc([N+](=O)[O-])cc2)c1C(N)=O ; O=C(c1ccc(F)cc1)c1ccc(-c2[nH]c(-c3ccc([N+](=O)[O-])cc3O)nc2-c2ccccc2)cc1 ; "and" ; Nc1nnc(SCC(=O)c2ccc([N+](=O)[O-])cc2)[nH]1]

15. The compound of claim 1, or a pharmaceutically acceptable salt thereof, having the formula: 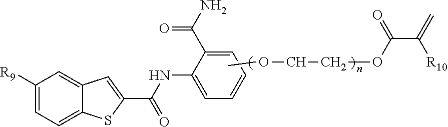
wherein R₉ is selected from the group of NO₂ and NH₂; R₁₀ is selected from the group of H and —CH₃; and n is an integer selected from the group of 1, 2, 3, 4, 5, 6, 7, 8, 9, 10, 11, 12, 13, 14, 15, 16, 17, 18, 19, and 20.

16. A compound of claim 1, or a pharmaceutically acceptable salt thereof, wherein the compound has the formula: 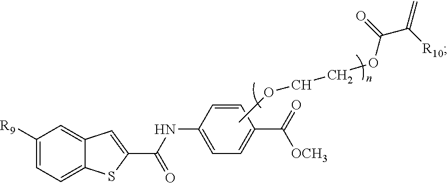
wherein R₉ is selected from the group of NO₂ and NH₂; R₁₀ is selected from the group of H and —CH₃; and n is an integer selected from the group of 1, 2, 3, 4, 5, 6, 7, 8, 9, 10, 11, 12, 13, 14, 15, 16, 17, 18, 19, and 20.

17. A compound of claim 1, or a pharmaceutically acceptable salt thereof, wherein the compound has the formula: 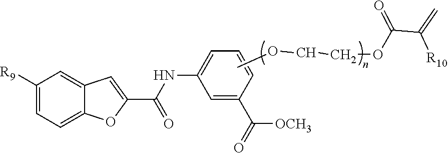
wherein R₉ is selected from the group of NO₂ and NH₂; R₂₀ is selected from the group of H and —CH₃; and n is an integer selected from the group of 1, 2, 3, 4, 5, 6, 7, 8, 9, 10, 11, 12, 13, 14, 15, 16, 17, 18, 19, and 20.

18. A compound of claim 1, or a pharmaceutically acceptable salt thereof, wherein the compound has the formula: 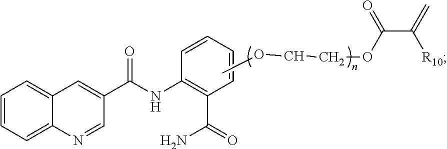
wherein R₁₀ is selected from the group of H and —CH₃; and n is an integer selected from the group of 1, 2, 3, 4, 5, 6, 7, 8, 9, 10, 11, 12, 13, 14, 15, 16, 17, 18, 19, and 20.

19. A compound of claim 1, or a pharmaceutically acceptable salt thereof, wherein the compound has the formula: 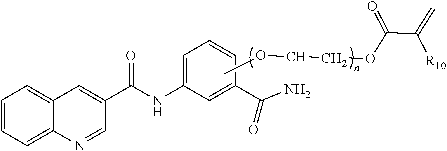
wherein R₁₀ is selected from the group of H and —CH₃; and n is an integer selected from the group of 1, 2, 3, 4, 5, 6, 7, 8, 9, 10, 11, 12, 13, 14, 15, 16, 17, 18, 19, and 20.

20. The compound of claim 1 having the structure: 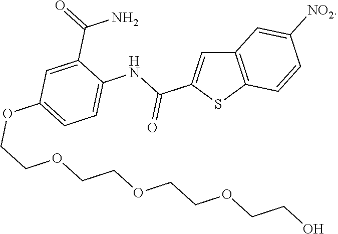 [SMILES: NC(=O)c1cc(OCCOCCOCCOCCO)ccc1NC(=O)c1cc2cc([N+](=O)[O-])ccc2s1]
